(19) United States
(12) Patent Application Publication
(10) Pub. No.: US 2006/0004291 A1
(43) Pub. Date: Jan. 5, 2006

(54) Methods and apparatus for visualization of quantitative data on a model

(76) Inventors: Andreas Heimdal, Oslo (NO); Stein Inge Rabben, Sofiemyr (NO); Arve Stavo, Tronheim (NO); Rune Torkildsen, Foreidgrend (NO); Ditlef Martens, Bergen (NO)

Correspondence Address: DEAN D. SMALL (12553 + 1000), ARMSTRONG TEASDALE LLP, ONE METROPOLITAN SQUARE, SUITE 2600, ST. LOUIS, MO, 63102-2740, US

(21) Appl. No.: 10/925,457

(22) Filed: Aug. 25, 2004

Related U.S. Application Data
(60) Provisional application No. 60/581,675, filed on Jun. 22, 2004.

(51) Int. Cl. A61B 8/12; A61B 8/00

(52) U.S. Cl. 600/459; 600/437

(57) ABSTRACT

An ultrasound method for visualization of quantitative data on a surface model is provided. The ultrasound method acquires ultrasound information from an object. The information acquired defines ultrasound images along at least first and second scan planes through the object and is stored in a buffer memory. The method then constructs a surface model of the object based on the ultrasound information. Timing information associated with local areas on the object is determined. The surface model and timing information are displayed with the timing information being positioned proximate regions of the surface model corresponding to local areas on the object.

### CROSS REFERENCE TO RELATED APPLICATIONS

This application claims priority to and the benefit of the filing date of U.S. Provisional Application No. 60/581,675 filed on Jun. 22, 2004 and which is hereby incorporated by reference in its entirety.

### BACKGROUND OF THE INVENTION

The present invention relates to diagnostic ultrasound methods and systems. In particular, the present invention relates to methods and systems for visualizing ultrasound data sets on a model.

Numerous ultrasound methods and systems exist for use in medical diagnostics. Various features have been proposed to facilitate patient examination and diagnosis based on ultrasound images of the patient. For example, certain systems offer various techniques for obtaining volume rendered data. Systems have been developed to acquire information corresponding to a plurality of two-dimensional representations or image planes of an object for three-dimensional reconstruction and surface modeling.

Heretofore, quantitative object time data has yet to be shown associated with the areas of a surface model. In the past, ultrasound methods and systems were unable to present quantitative time data with surface model rendering techniques.

A need exists for improved methods and systems that are able to implement surface model rendering techniques for the visualization of quantitative data.

### BRIEF DESCRIPTION OF THE INVENTION

An ultrasound method for visualization of quantitative data on a surface model is provided. The ultrasound method acquires ultrasound information from an object. The information acquired defines ultrasound images along at least first and second scan planes through the object and is stored in a buffer memory. The method then constructs a surface model of the object based on the ultrasound information. Timing information associated with local areas on the object is determined. The surface model and timing information are displayed with the timing information being positioned proximate regions of the surface model corresponding to local areas on the object.

In accordance with an alternative embodiment, an ultrasound system is provided that includes a probe to acquire ultrasound information from an object and a memory for storing the ultrasound information along at least first and second scan planes through the object. A processor for constructing a surface model of the object based on the ultrasound information is included. The processor determines timing information associated with local areas on the object. The system includes a display for presenting a surface model and the timing information, with the timing information being positioned proximate regions of the surface model corresponding to the local areas on the object.

### BRIEF DESCRIPTION OF THE DRAWINGS

FIG. 1 is a block diagram of an ultrasound system formed in accordance with an embodiment of the present invention.

FIG. 2 is a block diagram of an ultrasound system formed in accordance with an alternative embodiment of the present invention.

FIG. 3 is a flowchart of an exemplary method for mapping quantitative object timing information onto a surface model.

FIG. 4 illustrates an embodiment of a screen display of geometrical surface model showing tissue synchronization imaging (TSI) data.

FIG. 5 illustrates a top view of three scan planes through the surface model of FIG. 4 used to generate the surface model.

FIG. 6 shows a Bull's Eye plot that maps TSI data in accordance with an alternative embodiment of the present invention.

FIG. 7 illustrates an embodiment of a view of a mitral ring in which three data planes intersect to generate a visualization of a dynamic mitral valve (MV) ring.

FIG. 8 illustrates a resultant ring that may be constructed when a spline is fitted through two points in each of the three planes of FIG. 7.

FIG. 9 illustrates longitudinal movement of two points on the mitral ring of FIG. 8 and upward movement thereof in relation to each other.

### DETAILED DESCRIPTION OF THE INVENTION

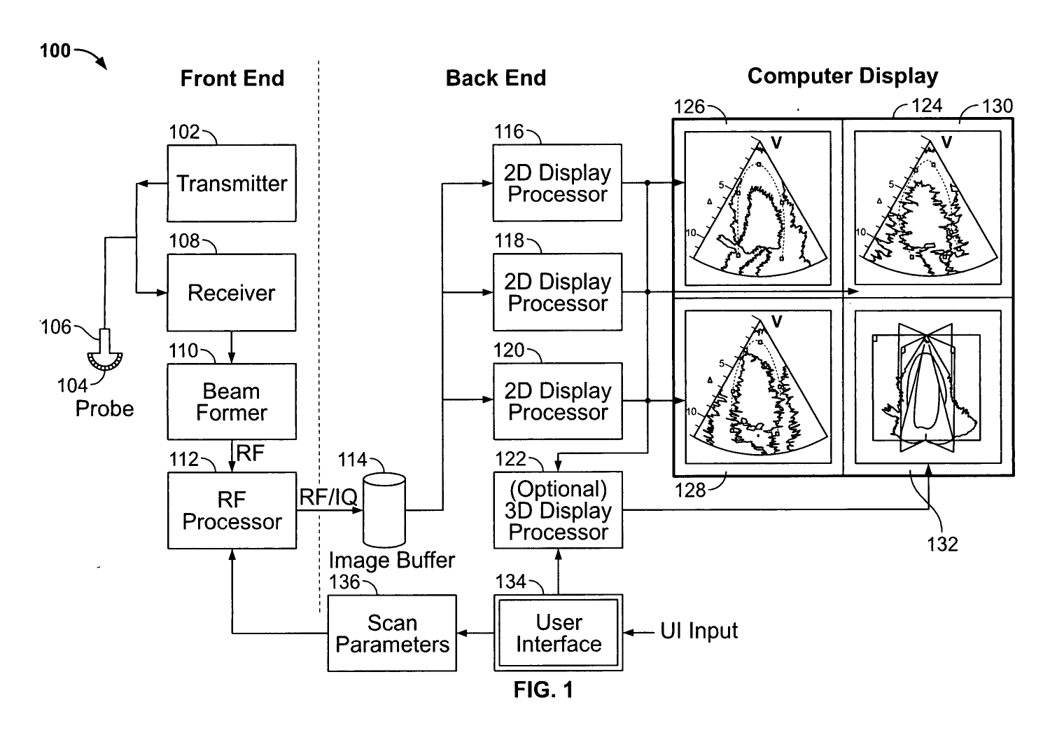

FIG. 1 is a block diagram of an ultrasound system 100 formed in accordance with an embodiment of the present invention. The ultrasound system 100 is configurable to acquire information corresponding to a plurality of two-dimensional (2D) representations or images of a region of interest (ROI) in a subject or patient. One such ROI may be the human heart or the myocardium of a human heart. The ultrasound system 100 is configurable to acquire 2D image planes in two or more different planes of orientation. The ultrasound system 100 includes a transmitter 102 that, under the guidance of a beamformer 110, drives a plurality of transducer elements 104 within an array transducer 106 to emit pulsed ultrasound signals into a body. The elements 104 within the array transducer 106 are excited by an excitation signal received from the transmitter 102 based on control information received from the beamformer 110. When excited, the transducer elements 104 produce ultrasonic waveforms that are directed along transmit beams into the subject. The ultrasound waves are back-scattered from density interfaces and/or structures in the body, like blood cells or muscular tissue, to produce echoes which return to the transducer elements 104. The echo information is received and converted into electrical signals by the transducer elements 104. The electrical signals are transmitted by the array transducer 106 to a receiver 108 and subsequently passed to the beamformer 110. In the embodiment described below, the beamformer 110 operates as a transmit and receive beamformer.

The beamformer 110 delays, apodizes and sums each electrical signal with other electrical signals received from the array transducer 106. The summed signals represent echoes from the ultrasound beams or lines. The summed signals are output from the beamformer 110 to an RF processor 112. The RF processor 112 may generate in phase and quadrature (I and Q) information. Alternatively, real value signals may be generated from the information received from the beamformer 110. The RF processor 112 gathers information (e.g. I/Q information) related to one frame and stores the frame information with time stamp and orientation/rotation information into an image buffer 114. Orientation/rotation information may indicate the angular rotation one frame makes with another. For example, in a tri-plane situation whereby ultrasound information is acquired simultaneously for three differently oriented planes or views, one frame may be associated with an angle of 0 degrees, another with an angle of 60 degrees, and a third with an angle of 120 degrees. Thus, frames may be added to the image buffer 114 in a repeating order of 0 degrees, 60 degrees, 120 degrees, . . . 0 degrees, 60 degrees, 120 degrees . . . . The first and fourth frame in the image buffer 114 have a first common planar orientation. The second and fifth frames have a second common planar orientation and the third and sixth frames have a third common planar orientation. Alternatively, in a biplane situation, the RF processor 112 may collect frame information and store the information in a repeating frame orientation order of 0 degrees, 90 degrees, 0 degrees, 90 degrees, . . . . The frames of information stored in the image buffer 114 are processed by the 2D display processor 116. Other acquisition strategies may include multi-plane variations of interleaving and frame rate decimation . . . . Also rotation of multi-plane to get higher spatial resolution by combining several beats.

The 2D display processors 116, 118, and 120 operate alternatively and successfully in round-robin fashion processing image frames from the image buffer 114. For example, the display processors 116, 118, and 120 may have access to all of the data slices in the image buffer 114, but are configured to operate upon data slices having one angular orientation. For example, the display processor 116 may only process image frames from the image buffer 114 associated with an angular rotation of 0 degrees. Likewise, the display processor 118 may only process 60 degree oriented frames and the display processor 120 may only process 120 degree oriented frames.

The 2D display processor 116 may process a set of frames having a common orientation from the image buffer 114 to produce a 2D image or view of the scanned object in a quadrant 126 of a computer display 124. The sequence of image frames played in the quadrant 126 may form a cine loop. Likewise, the display processor 118 may process a set of frames from the image buffer 114 having a common orientation to produce a second different 2D view of the scanned object in a quadrant 130. The display processor 120 may process a set of frames having a common orientation from the image buffer 114 to produce a third different 2D view of the scanned object in a quadrant 128.

For example, the frames processed by the display processor 116 may produce an apical 2-chamber view of the heart to be shown in the quadrant 126. Frames processed by the display processor 118 may produce an apical 4-chamber view of the heart to be shown in the quadrant 130. The display processor 120 may produce frames to form an apical parasternal long-axis (PLAX) view of the heart to be shown in the quadrant 128. All three views of the human heart may be shown simultaneously in real time in the three quadrants 126, 128, and 130 of the computer display 124.

A 2D display processor, for example the processor 116, may perform filtering of the frame information received from the image buffer 114, as well as processing of the frame information, to produce a processed image frame. Some forms of processed image frames may be B-mode data (e.g. echo signal intensity or amplitude) or Doppler data. Examples of Doppler data include color flow data, color power Doppler), or Doppler Tissue data. The display processor 116 may then perform scan conversion to map data from a polar to Cartesian coordinate system for display on a computer display 124.

Optionally, a 3D display processor 122 may be provided to process the outputs from the other 2D display processors 116, 118, and 120. Processor 122 may combine the 3 views produced from 2D display processors 116, 118, and 120 to form a tri-plane view in a quadrant 132 of the computer display 124. The tri-plane view may show a 3D image, e.g. a 3D image of the human heart, aligned with respect to the 3 intersecting planes of the tri-plane. In one embodiment, the 3 planes of the tri-plane intersect at a common axis of rotation.

A user interface 134 is provided which allows the user to input scan parameters 136. The scan parameters 136 may allow the user to designate what number of planes in the scan is desired. The scan parameters may allow for adjusting the depth and width of a scan of the object for each of the planes of the tri-plane. When performing simultaneous acquisition of scan data from three planes, the beamformer 110 in conjunction with the transmitter 102 signals the array transducer 106 to produce ultrasound beams that are focused within and adjacent to the three planes that slice the scan object. The reflected ultrasound echoes are gathered simultaneously to produce image frames that are stored in the image buffer 114. As the image buffer 114 is being filled by the RF processor 112, the image buffer 114 is being emptied by the 2D display processors 116, 118, and 120. The 2D display processors 116, 118, and 120 form the data for viewing as 3 views of the scan object in corresponding computer display quadrants 126, 130, and 128. The display of the 3 views in quadrants 126, 130, and 128, as well as an optional displaying of the combination of the 3 views in quadrant 132, is in real time. Real time display makes use of the scan data as soon as the data is available for display.

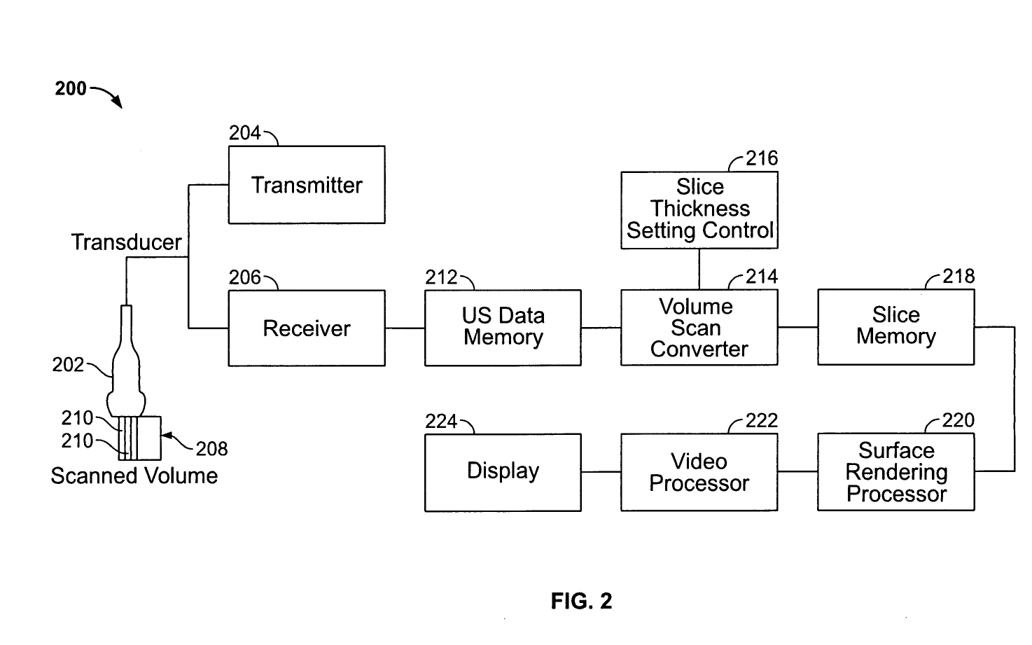

FIG. 2 is a block diagram of an ultrasound system 200 formed in accordance with an alternative embodiment of the present invention. The system includes a probe 202 connected to a transmitter 204 and a receiver 206. The probe 202 transmits ultrasonic pulses and receives echoes from structures inside of a scanned ultrasound volume 208. The memory 212 stores ultrasound data from the receiver 206 derived from the scanned ultrasound volume 208. The volume 208 may be obtained by various techniques (e.g., 3D scanning, real-time 3D imaging, volume scanning, 2D scanning with transducers having positioning sensors, freehand scanning using a voxel correlation technique, 2D or matrix array transducers and the like).

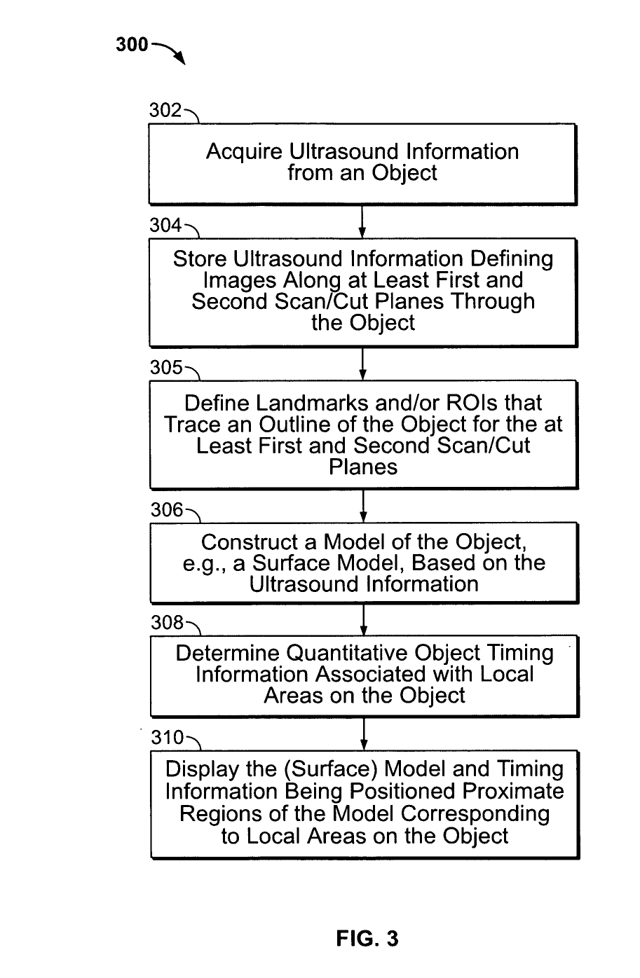

The probe 202 is moved, such as along a linear or arcuate path, while scanning a region of interest (ROI). At each linear or arcuate position, the probe 202 obtains scan planes 210. Alternatively, a matrix array transducer probe 202 with electronic beam steering may be used to obtain the scan planes 210 without moving the probe 202. The scan planes 210 are collected for a thickness, such as from a group or set of adjacent scan planes 210. The scan planes 210 are stored in the memory 212, and then passed to a volume scan converter 214. In some embodiments, the probe 202 may obtain lines instead of the scan planes 210, and the memory 212 may store lines obtained by the probe 202 rather than the scan planes 210. The volume scan converter 214 may process lines obtained by the probe 202 rather than the scan planes 210. The volume scan converter 214 receives a slice thickness setting from a control input 216, which identifies the thickness of a slice to be created from the scan planes 210. The volume scan converter 214 creates a 2D frame from multiple adjacent scan planes 210. The frame is stored in slice memory 218 and is accessed by a surface rendering processor 220. The surface rendering processor 220 performs surface rendering upon the frame at a point in time by performing an interpolation of the values of adjacent frames. The output of the surface rendering processor 220 is passed to the video processor 222 and the display 224. The position of each echo signal sample (voxel) is defined in terms of geometrical accuracy (i.e., the distance from one voxel to the next) and ultrasonic response (and derived values from the ultrasonic response). Suitable ultrasonic responses include gray scale values, color flow values, tissue velocity, strain rate and angio or power Doppler information, and combinations thereof. B-mode data may be utilized to outline the model. The surface of the model is defined through surface rendering. Once the surface of the model is defined, quantitative information is then mapped onto the surface. The mapping operation may be achieved between frames by interpolation of adjacent frames or planes at different depths in first and second scan planes that intersect with one another along a common axis. FIG. 3 is a flowchart 300 of an exemplary method for mapping quantitative object timing information onto a model, e.g. a surface model, of the scanned object. At 302, ultrasound scan information is acquired from an object. For example, the object may constitute the heart and ultrasound scan information may be acquired to produce a cine loop of frames that are gathered over at least one heart cycle.

At 304, acquired ultrasound information is stored in a data memory 212 (FIG. 2) that defines images of the object along at least first and second scan planes through the object. The stored information includes multiple images that are associated within a single scan plane through the object at different points in time, and multiple images from different scan planes at a common point in a cyclical motion of the object. In the example of first and second scan planes (a biplane scan), first and second scan planes may intersect with one another along a common axis through the object whereby the planes are oriented at a predetermined angle with respect to one another. Likewise, for an the example of three planes (a tri-plane scan), the three planes may intersect with one another along a common axis through the object whereby the planes are oriented at a predetermined angle with respect to one another.

At 306, a model, for example a 3D surface model, a bull's eye model, or a heart mitral valve (MV) ring model is constructed of the object based on the ultrasound information acquired. An outline of the object may be manually determined by mouse clicking on a series of points (or mouse drawing of contours) in one of the planar images of the object at predefined times in the cyclical motion of the object. The mouse may be part of the user interface 134. A manual determination of the outline may be done in each of the three data planes. In an alternative embodiment, the outline of the object/landmark may be determined automatically within the data planes by the 3D display processor 122 of FIG. 1 detecting boundaries or landmarks of the object, such as for the example of a human heart, the AV plane, the mitral ring, and the apex.Color may be used to indicate the degree of time delay in TSI data. A color is assigned automatically by the system 100 to each designated ROI in the three frames depending on the movement of that particular ROI over time. A color may be associated with a ROI that indicates the time from a reference time of the ROI to a peak velocity. For example, the interval chosen for real time measurement of time delay may be from the time of QRS in the cardiac cycle to the time of peak tissue velocity within a given search interval. The search interval may, for example, be from aortic valve opening time to aortic valve closure time. The color assigned to each ROI is based on the color scale and the time to peak velocity for the ROI over time. For, example, an ROI having a short time delay is assigned a green color, an ROI having a medium time delay is assigned a yellow color, and an ROI having a long time delay is assigned a reddish orange color. Therefore, green indicates tissue with early motion and red indicates tissue with delayed motion. The color is mapped onto a gray scale image of each frame such that a particular color corresponds to an intensity level or brightness of the grayscale or B-mode frame. Once the color mapping of the grayscale image is accomplished, the system 100 automatically interpolates the colors of the three frames onto the surface model as it is generated.

At 308, quantitative object timing information, such as one of tissue velocity, displacement, strain, and strain rate, associated with local areas on the object is determined. The object timing information associated with the local areas is relative to a reference time for the local areas. In the example of the human heart, the reference time may be the QRS point in the heart cycle. The timing information defines a time from the reference time to when the local area reaches a particular state in a series of states through which the object cycles. Quantitative object timing information may be used to detect malfunctioning of tissues of the object. For example, an area of tissue may be found in the human heart to lag in time from QRS to reach peak velocity in contrast to surrounding tissue areas. The lag in time to reach peak velocity may indicate the presence of diseased tissue.

At 310, a model, e.g. a 3D surface model, bull's eye surface model, or mitral valve ring surface model, and object timing information are displayed. The timing information being displayed is positioned proximate regions of the model corresponding to the local areas on the object. The timing information may constitute at least one of color coding of, and vector indicia on, the regions of the model. Color coding with a range of colors indicating a range from normal to abnormal may be used to visualized the desired parameter of quantitative object timing data, e.g. time to peak tissue velocity, time to peak strain. Such color coding may visually identify asynchronous areas of tissue, for example in the heart, that are unhealthy.

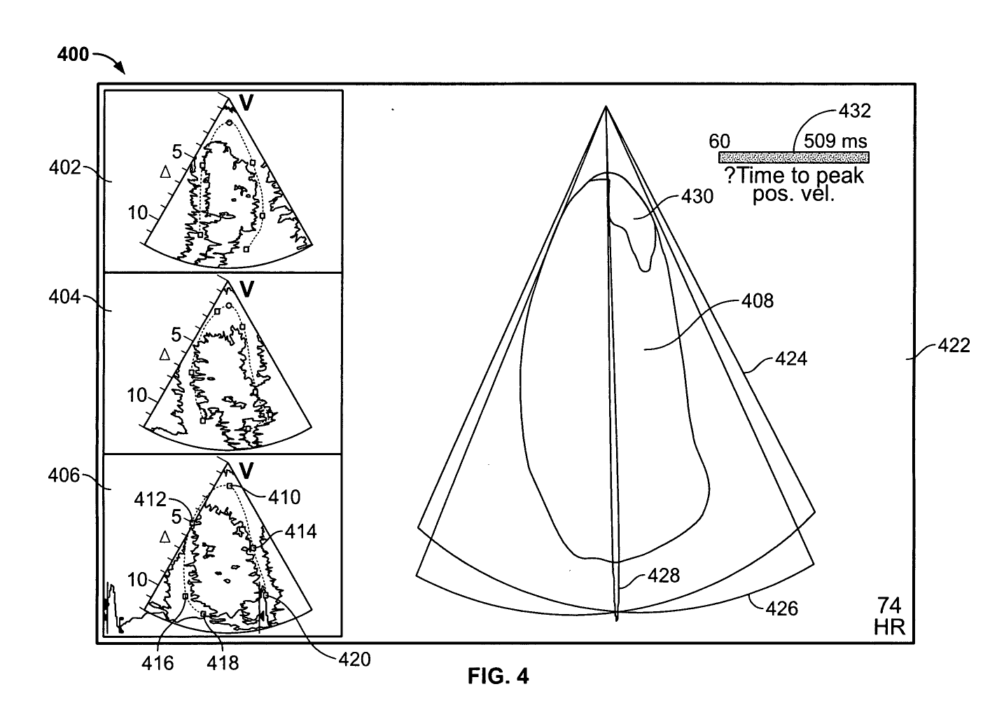

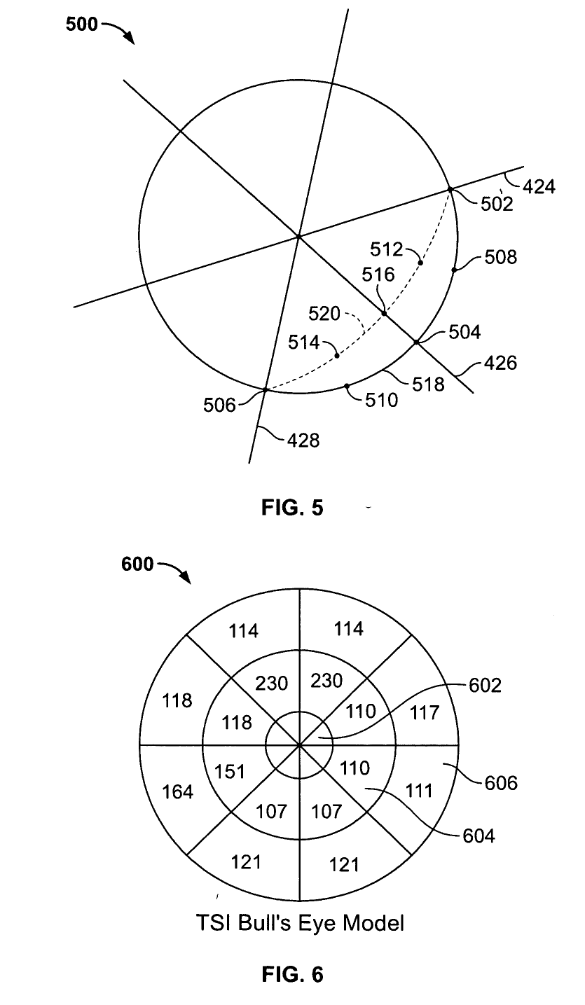

FIG. 4 illustrates an embodiment of a screen display 400 of a geometrical surface model 408 showing tissue synchronization imaging (TSI) data to indicate tissue motion delay. The display 400 may be shown on the computer display screen 124 of FIG. 1. The three data planes 402, 404, and 406 shown in the left side of the figure are based on the ultrasound information acquired 302 and stored 304. The surface model 408 may be generated automatically from the data planes 402, 404, and 406. The user may manually trace an outline of the object by defining/marking 305 landmarks or ROIs, for example ROIs 410, 412, 414, 416, 418, and 420 are designated in data plane 406 by a user mouse clicking on these areas. Likewise, landmarks and/or ROIs may be defined 305 to outline the object in frames or planes 402 and 404. In an alternative embodiment, the system 100 may automatically define 305 the landmarks and/or ROIs from the ultrasound boundary interface data. For example, the system 100 of FIG. 1 may detect the AV plane and apex points at a given time and make a spline surface through these points. Some shape factors such as wideness and skew may be included. Once the outlines are defined 305 in all three data planes 402-406, the system 100 generates or constructs 306 the surface model 408. The three data planes 402, 404, and 406 are correspondingly shown in the surface model view 422 as planes 424, 426, and 428. In order to construct 306 the surface model 408, interpolation of the data in the planes 424, 426, and 428 is done to determine the points between the planes through which the constructed surface model 408 may be drawn. The contour of the surface of the object depends on the shape of the connected points. Depending where a landmark or ROI is manually or automatically determined, the contour may be circular or not, as shown in FIG. 5.

The geometrical surface model 408 may be color coded to determine 308 and to visually display 310 the mapping of quantitative object timing data. For example, a portion of the surface model 408, for example portion 430, may be colored red-orange, while the rest of the surface outline is colored green. The color coding of the surface model 408 may indicate the portion 430 having a reddish-orange color for mapped TSI data which indicates tissue with delayed motion, while the rest of the surface is colored in green to indicate tissue with early motion.

FIG. 5 illustrates a top view 500 of the three scan planes 424, 426, and 428 of FIG. 4. Landmark or ROI points 502, 504, and 506 may be manually defined 305 by the user as described for the ROIs 410-420 of FIG. 4, or automatically determined by the system 100. A point 508 may be interpolated from the planar points 502 and 504 by the system 100. Likewise, a point 510 may be interpolated from the planar points 504 and 506 by the system 100. The points 502, 508, 504, 510 and 506 may when connected by the system 100 generate a circular contour 518. Alternatively, if ROI point 516 is selected instead of ROI point 504 by the user, the points 512 and 514 may be interpolated in contrast to the corresponding points 508 and 510. System 100 in this case may generate a non-circular contour 520 through the points 502, 512, 516, 514, and 506. In a similar manner, the complete surface model 408 may be generated/constructed 306 by the system 100 from points selected in the planes 424-428 and points interpolated between the planes.

Color coding is accomplished according to 308 and 310 in FIG. 3. The time interval chosen for the embodiment of FIG. 4 is the time to peak velocity. Therefore, the interval chosen for real-time measurement of time delay is from the time of QRS in the cardiac cycle to the time of peak tissue velocity. A color scale 432 is shown with a color gradient ranging from green to yellow to red. At the one end of the scale, the color green corresponds to a short time delay starting at 60 milliseconds (ms). The other end of the scale is red and indicates a long time delay extending to a maximum of 509 ms. FIG. 6 shows the generation of a Bull's Eye plot 600 that maps TSI data. The Bull's eye is a bottom or apical view of the heart that is projected onto a flat or 2D surface model or a plane. The center 602 of the Bull's eye where the crosshair is located is the apex of the heart. The middle ring area 604 is the middle segments of the left ventricle of the heart. The outer ring area 606 is the basal segments of the heart and includes the mitral valve ring. The numbers on the Bull's eye plot 600 represent quantitative data on time to peak velocity after QRS complex in different regions of the ventricle. In FIG. 6, the value 230 represents the area of maximum time to peak tissue velocity. Asynchrony in terms of TSI calculated indexes derived for these numbers include, septal lateral delay, septal posterior delay, maximum delay, standard deviation, etc. The numbers may be generated automatically as for a geometric surface model or they may be based on manual positioning of sampling regions. Manual measurement of time to time to peak velocity is determined 308 by single-clicking in each (basal and mid) segment. The result is presented or displayed 310 in a Bull's eye plot 600 as color coding and numbers therein. The numbers on the Bull's eye plot may be automatically generated or determined 308 as a function of the velocity measurements in different areas of the heart.

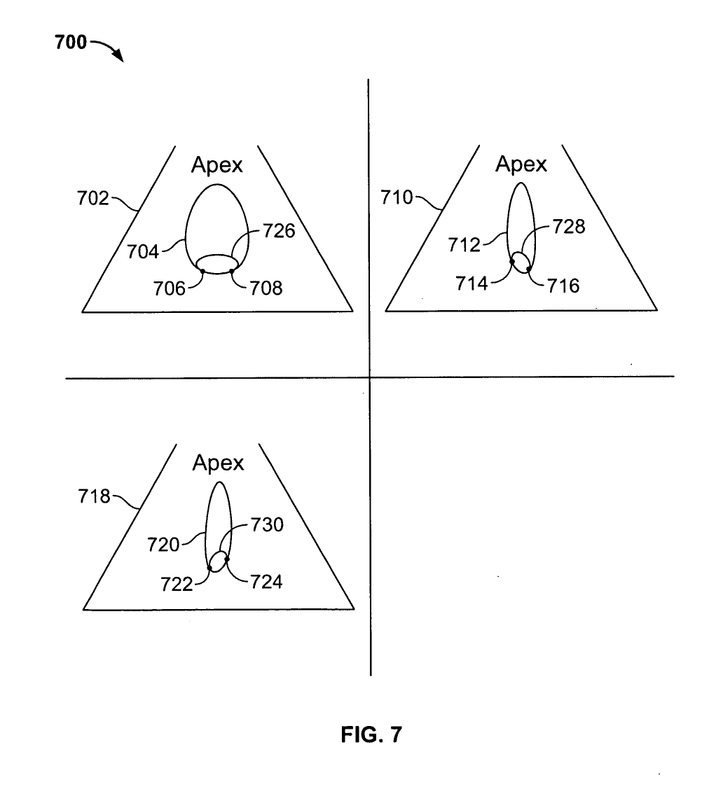

FIG. 7 illustrates an embodiment of a view 700 of a mitral ring in which three data planes 702, 710, and 718 intersect the mitral ring valve at different angles to one another to generate a visualization of a dynamic mitral valve (MV) ring. The position of the mitral ring may be tracked throughout a whole heart cycle in a 3D data set. Then the tracking is visualized as a model which is an integrated image of all the mitral ring positions throughout the cycle. This can be used to extract information on heart function. The model is cylindrical for a heart where the walls are well-synchronized. The initial position of the mitral valve ring may either be manually defined or detected automatically by an AV-plane detector. Two points in each plane through the left ventricle may be selected in each of the three data planes of the left ventricle 704, 712, and 720 in FIG. 7. In plane 702, the points 706 and 708 are chosen on the mitral valve ring 726 of the left ventricle 704. In plane 710, the points 714 and 716 are chosen on the mitral valve ring 728 of the left ventricle 712. In plane 718, the points 722 and 724 are chosen on the mitral valve ring 730 of the left ventricle 720. The six points 706, 708, 714, 716, 722, and 724 are connected as shown in FIG. 8.

FIG. 8 illustrates the resultant ring 800 that may be constructed when a spline is fitted through the six points 706, 708, 714, 716, 722, and 724 of FIG. 7. Upon detecting the location of the AV plane and the mitral ring and with the use of the apex, these three landmarks in the three planes may be used to construct an outline of the left ventricle. The MV ring 800, which lies in the AV plane, may be visualized by looking at sequential sets of the six points over time. The longitudinal or up and down motion of the ring along the long axis of the ventricle in relation to the apex may be viewed over time as an index of synchronicity. The index of synchronicity of the points along the mitral valve ring structure may be used to obtain a dynamic view of the mitral ring valve function.

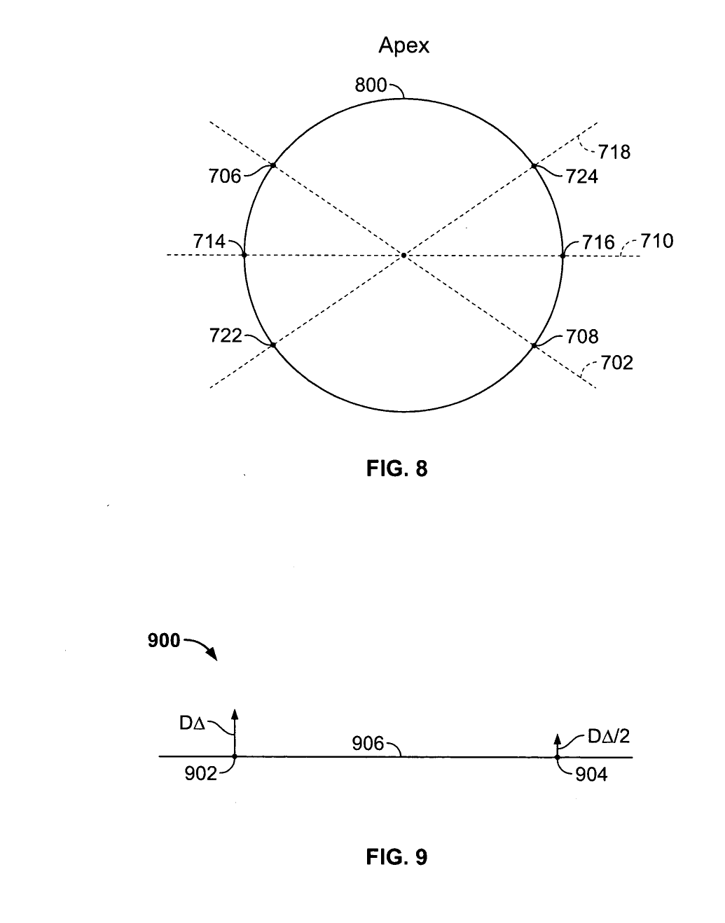

FIG. 9 illustrates longitudinal movement 900 of two points 902 and 904 on the mitral ring of FIG. 8 and their upward movement in relation to each other. Point 902 may move a distance of delta, whereas point 904 may move a lesser distance of only delta over two in relation to point 902. After the dynamic mitral ring model 800 is generated, several M&A parameters may be extracted from the model data. The degree of asynchrony may (TSI), excursion of the six standard apical segments or E′ wave velocity of the six standard apical segments (relaxation) may be determined.

The tissue delay of different regions of interest may be compared to identify a degree of delay. For example, ROIs corresponding to segments within the left ventricle may be compared to identify the most delayed segment. Similarly, segments may be compared between the left and right ventricles. Although FIG. 4 illustrates data representative of a left ventricle, thus quantifying the amount of synchrony within the left ventricle, it should be understood that the method of FIG. 3 may be used to quantify and compare the time delay of samples in the left and right ventricles. In this way, the amount of synchrony between the left and right ventricles may be determined. In addition, the method of FIG. 3 may be used to quantify and compare the time delay of regions in the left and right atria of the heart.

The system and method described herein include automatic detection of peaks, zero-crossings or other features of tissue velocity, displacement, strain rate and strain data as a function of time. By processing only the image frames within the selected time interval, the processing time is shortened and the possibility of false positives is lowered, such as may occur when an incorrect peak is identified. The system and method color codes the delay of samples in the image in relation to the onset of the QRS, and presents the data as a parametric image, both in live display and in replay. Thus, heart segments or other selected tissue with delayed motion might be more easily visualized than with other imaging modes. Therefore, patients who will respond favorably to cardiac resynchronization therapy (CRT) may be more easily selected, and the optimal position for the left ventricle pacing lead for a cardiac pacemaker may be located by identifying the most delayed site within the left ventricle. Furthermore, the effect of the various pacemaker settings, such as AV-delay and VV-delay, may be studied to find the optimal settings.

Optionally, the model may not be a surface model. Instead, the model may be a splat rendering, Bulls-Eye, a mitral ring model and the like.

While the invention has been described in terms of various specific embodiments, those skilled in the art will recognize that the invention can be practiced with modification within the spirit and scope of the claims.

What is claimed is:

1. An ultrasound system comprising:
a probe for acquiring ultrasound information from an object;
memory storing said ultrasound information defining ultrasound images along at least first and second scan planes through the object; a processor constructing a model of the object based on the ultrasound information, said processor determining timing associated with local areas on the object; and
a display presenting said model and said timing information, said timing information being positioned proximate regions of said model corresponding to the local areas on the object.

2. The ultrasound system of claim 1, wherein said processor includes at least first and second processors that process ultrasound information associated with first and second scan planes, respectively.

3. The ultrasound system of claim 1, wherein said memory includes a scan buffer storing multiple ultrasound images associated with a single scan plane through the object at different points in time.

4. The ultrasound system of claim 1, wherein said memory includes a scan buffer storing multiple ultrasound images associated with said at least first and second scan planes rotated during multi-plane acquisition.

5. The ultrasound system of claim 1, wherein said first and second scan planes intersect with one another along a common axis through the object and are oriented at a predetermined angle with respect to one another.

6. The ultrasound system of claim 1, wherein said processor performs interpolation based on said ultrasound images in first and second scan planes to derive synthetic ultrasound data estimating timing information on each point of the model.

7. The ultrasound system of claim 1, further comprising an input configured to permit a user to manually identify an outline of the object at predefined times in a cyclical motion of the object, said processor constructing said surface model based on said outline of the object.

8. The ultrasound system of claim 1, wherein said timing information constitutes at least one of color coding of, and a number on, said regions of said model.

9. The ultrasound system of claim 1, wherein said processor determines said timing information for said regions relative to a reference time, for each of said regions, said timing information defining a time from said reference time to when said region reaches a particular state in a series of states through which the object cycles.

10. The ultrasound system of claim 1, wherein the object constitutes the heart and said displays a cine loop of at least one heart cycle, said timing information identifying said regions of the heart that have early motion in a first color and delayed motion in a second color.

11. An ultrasound method, comprising:
acquiring ultrasound information from an object;
storing said ultrasound information defining ultrasound images along at least first and second scan planes through the object;
constructing a model of the object based on the ultrasound information;
determining timing information associated with local areas on the object; and
displaying said model and said timing information, said timing information being positioned proximate regions of said model corresponding to the local areas on the object.

12. The ultrasound method of claim 11, wherein said timing information is derived based on at least one of tissue velocity imaging, tissue displacement imaging, strain, and strain rate imaging.

13. The ultrasound method of claim 11, wherein said storing includes storing multiple ultrasound images associated with a single scan plane through the object at different points in time.

14. The ultrasound method of claim 11, wherein said storing includes storing multiple ultrasound images associated with said at least first and second scan planes rotated during multi-plane acquisition.

15. The ultrasound method of claim 11, wherein said first and second scan planes intersect with one another along a common axis through the object and are oriented at a predetermined angle with respect to one another.

16. The ultrasound method of claim 11, further comprising interpolating, based on said ultrasound images in first and second scan planes, to derive synthetic ultrasound data estimating timing information on each point of the model.

17. The ultrasound method of claim 11, further comprising permitting a user to manually identify an outline of the object at predefined times in a cyclical motion of the object, and constructing said surface model based on said outline of the object.

18. The ultrasound method of claim 11, wherein said timing information constitutes at least one of color coding of, and a number on, said regions of said model.

19. The ultrasound method of claim 11, further comprising determining said timing information for said regions relative to a reference time, for each of said regions, said timing information defining a time from said reference time to when said region reaches a particular state in a series of states through which the object cycles.

20. The ultrasound method of claim 11, wherein the object constitutes the heart and said display displays a cine loop of at least one heart cycle, said timing information identifying said regions of the heart that have early motion in a first color and delayed motion in a second color.
